(19) United States
(12) Patent Application Publication
(10) Pub. No.: US 2021/0042029 A1
(43) Pub. Date: Feb. 11, 2021

(54) ENGLISH INPUT KEYBOARD FOR SEVERE PATIENT

(71) Applicant: Hyun Jin YUN, Seoul (KR)

(72) Inventor: Hyun Jin YUN, Seoul (KR)

(21) Appl. No.: 16/984,355

(22) Filed: Aug. 4, 2020

(30) Foreign Application Priority Data
Aug. 5, 2019 (KR) 1020190094912

(51) Int. Cl. G06F 3/0488; G06F 3/0484; G06F 3/01

(52) U.S. Cl. CPC G06F 3/04886; G06F 3/013; G06F 3/04847

(57) ABSTRACT

An English input keyboard for a severe patient includes an integrated dial in which all alphabets and numbers used in English are disposed to be divided into a predetermined number in multiple divided display windows, and a detail dial in which an alphabet and number included in the divided display window are disposed. A patient selects a window showing intended information on the English input keyboard presented through a movement of a pupil of the patient, and a guardian can communicate with the patient by combining the selected alphabets.

### CROSS REFERENCE TO RELATED APPLICATION

The present application claims Paris Convention priority from, and the benefit under U.S. law and/or all other applicable law to, Korean Patent Application No. 10-2019-0094912 entitled English Input Keyboard for Severe Patient filed on Aug. 5, 2019, the contents of which are incorporated herein by reference for all purposes.

### BACKGROUND OF THE DISCLOSURE

### Field of the Disclosure

disclosure relates to an English input keyboard for a severe patient, and more particularly, to an English input keyboard which can be used by a patient who suffers from a disease such as a Lou Gehrig's disease and is capable of performing mental activity but can hardly move a his/her body and move only his/her eyes.

### Related Art

In general, complementary and alternative communication for the disabled with communication is mainly a technology for children with developmental disabilities, and a product for children with developmental disabilities who have not restrictions on exercise abilities dominates. However, an adult patient with a motor neuron disorder such as Amyotrophic Lateral

Sclerosis (ALS), Kennedy Disease (KD), and Progressive Bulbar Palsy (PBP) has progressively limited motor skills, and thus, reaches a situation in which language communication is very difficult.

Most of a body of the patient gradually lose an ability to exercise, and finally, only eye gaze or eye movement is possible, which makes it almost impossible to communicate through words. In the end, eye gaze and eye movement are only signals for communication with the outside. However, in the related art, a product related to complementary and alternative communication considering special circumstances of the patient has not been developed.

In recent years, a technology such as a Brain-Computer Interface (BCI) which allows a user to express an intention of the user through a device has been developed. In this technology, the intension of the user is ascertained by analyzing the brain waves generated from a brain activity process in real time, and the intention is input to a keyboard or operate a specific device. In this way, a success of commercialization increases.

However, the BCI system is relatively expensive, and thus, is somewhat burdensome for a general user to encounter. Moreover, even if the BCI system is used, there is a cumbersome problem that the device for analyzing the brain waves should be always worn on a head of the user for simple communication. Accordingly, there is an increasing need to develop a product which can be easily used by a general user and allows a severe patient to easily perform communication.

### SUMMARY OF THE DISCLOSURE

The present disclosure provides an intermediary means which allows a severe patient, who suffers from a motor neuron disorder such as Amyotrophic Lateral Sclerosis (ALS), Kennedy Disease (KD), or Progressive Bulbar Palsy (PBP) and is unable to move most of the body, to easily perform a communication.

The present disclosure also provides an English input keyboard which can communicate with a guardian more quickly and conveniently when a thinking language of the severe patient is English.

In an aspect, there is provided an English input keyboard for a severe patient, including: an integrated dial in which all alphabets and Arabic numbers used in English are disposed to be divided into a predetermined number in multiple divided display windows; and a detail dial in which an alphabet and number included in the divided display window are disposed in one detail dial, in which the divided display window is disposed to have a predetermined angle inside the integrated dial, a plurality of sectors including the alphabet or number included in the divided display window are disposed in the detail dial to have a predetermined angle, and the number and positions of divided display windows inside the integrated dial and the number and positions of sectors inside the detailed dial are the same as each other, and a conversation partner of the patient presents the integrated dial to the patient, ascertains the divided display window in the integrated dial corresponding to a direction in which a pupil of the patient gazes, presents the detail dial including information in the divided display window to the patient, generates a word or a sentence by repeating a series of processes inputting the alphabet or the number of the sector corresponding to the direction in which the pupil of the patient gazes, and communicates with the patient.

Here, the integrated dial may further include a character input window where the conversation partner of the patient inputs the alphabet or number selected by the patient.

Moreover, six alphabets, six numbers, or six alphabets and numbers may be combined inside the divided display window, and based on a center of the integrated dial, the divided display window may include a first divided display window positioned at 12 o'clock, a second divided display window positioned at 10 o'clock, a third divided display window 130 positioned at 2 o'clock, a fourth divided display window positioned at 8 o'clock, ae fifth divided display window 150 positioned at 4 o'clock, and the sixth divided display window 160 positioned at 6 o'clock.

In this case, the English input keyboard for a severe patient may further include a first detailed dial in which character information included in the first divided display window is disposed one by one in six directions; a second detailed dial in which character information included in the second divided display window is disposed one by one in six directions; a third detailed dial in which character information included in the third divided display window is disposed one by one in six directions; a fourth detailed dial in which character information included in the fourth divided display window is disposed one by one in six directions; a fifth detailed dial in which character information included in the fifth divided display window is disposed one by one in six directions; and a sixth detailed dial in which character information included in the sixth divided display window is disposed one by one in six directions.

Moreover, the first detail dial to the sixth detail dial may include a first sector which is positioned at 12 o'clock based on a center and includes one character, a second sector which is positioned at 10 o'clock based on the center and includes one character, a third sector which is positioned at 2 o'clock based on the center and includes one character, a fourth sector which is positioned at 8 o'clock based on the center and includes one character, a fifth sector which is positioned at 4 o'clock based on the center and includes one character, and a sixth sector which is positioned at 6 o'clock based on the center and includes one character.

### BRIEF DESCRIPTION OF THE DRAWINGS

FIG. 1 is a diagram illustrating an integrated screen in an English input keyboard for a severe patient according to an embodiment of the present disclosure.

FIG. 2 is a diagram illustrating a detailed screen in the English input keyboard for a severe patient according to the embodiment of the present disclosure.

FIG. 3 is a diagram illustrating a process of inputting a character in the English input keyboard for a severe patient according to the embodiment of the present disclosure.

### DESCRIPTION OF EXEMPLARY EMBODIMENTS

Hereinafter, the present embodiment will be described in detail with reference to the accompanying drawings, but is not limited by the present embodiment. In describing the present disclosure, specific descriptions by known functions or configurations may be omitted to clarify a substance of the present disclosure.

The present disclosure relates to an English input keyboard which can be used by a patient with degenerative communication disorders and only eye gaze is possible in movements of a body of the patient. The English input keyboard may be provided as an application installed on a smartphone or pad, or as a set of plurality of dials, and hereinafter, an example of using an English input keyboard on the screen of an electronic device such as a smartphone will be described. In addition, the English input keyboard of the present disclosure is based on a typing method based on the English alphabet.

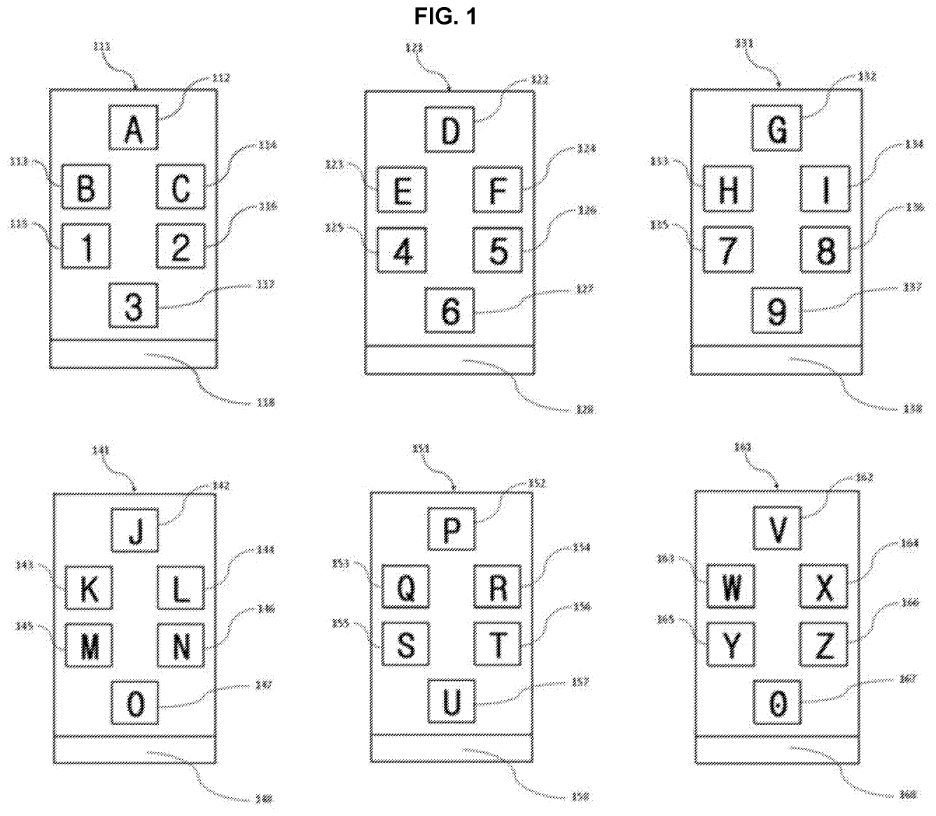

FIG. 1 is a diagram illustrating an integrated dial in an English input keyboard for a severe patient according to an embodiment of the present disclosure. Referring to FIG. 1, the integrated dial 100 includes all alphabets and all Arabic numerals constituting English and may be an English input keyboard first seen by a patient.

The integrated dial 100 may include a plurality of divided display windows 110, 120, 130, 140, and 150, a restart window 160, and a character input window 170. The divided display windows 110, 120, 130, 140, and 150 and the restart window 160 may be windows selected by eye gaze of a patient, and the character input window 170 may be a window input by a conversation partner (guardian) of the patient.

The divided display windows 110, 120, 130, 140, and 150 may be disposed according to a predetermined angle based on a center of the integrated dial 100. A total of 26 alphabets and 10 Arabic numerals constituting the English language may be divided into a predetermined number and displayed on respective divided display window.

In the embodiment, each of the divided display windows 110, 120, 130, 140, and 150 is disposed to include up to six alphabets and numbers, and the total number of alphabets and numbers is 36, and thus, the integrated dial 100 has a total of six divided display windows.

Based on the center of the integrated dial 100, the divided display windows 110, 120, 130, 140, and 150 may include the first divided display window 110 positioned at 12 o'clock, the second divided display window 120 positioned at 10 o'clock, the third divided display window 130 positioned at 2 o'clock, the fourth divided display window 140 positioned at 8 o'clock, the fifth divided display window 150 positioned at 4 o'clock, and the sixth divided display window 160 positioned at 6 o'clock. In the embodiment, each of the six divided display windows is spaced apart by a predetermined distance so that the gaze of the patient's eyes is divided into six directions and is disposed to have a predetermined angle with respect to the center.

The character input window 170 is an area where a patient's conversation partner (guardian) performs input, and may complete a word by inputting a character selected by the patient.

In FIG. 1, for convenience, the alphabets and numbers are arranged in the order of reading to be displayed on each divided display window. However, 26 alphabets and 10 numbers may be randomly mixed in each divided display window. The guardian may ascertain an alphabet or a number with a high frequency in selection of the patient to randomly dispose the alphabets and numbers for easy reading according to the eye gaze of the patient.

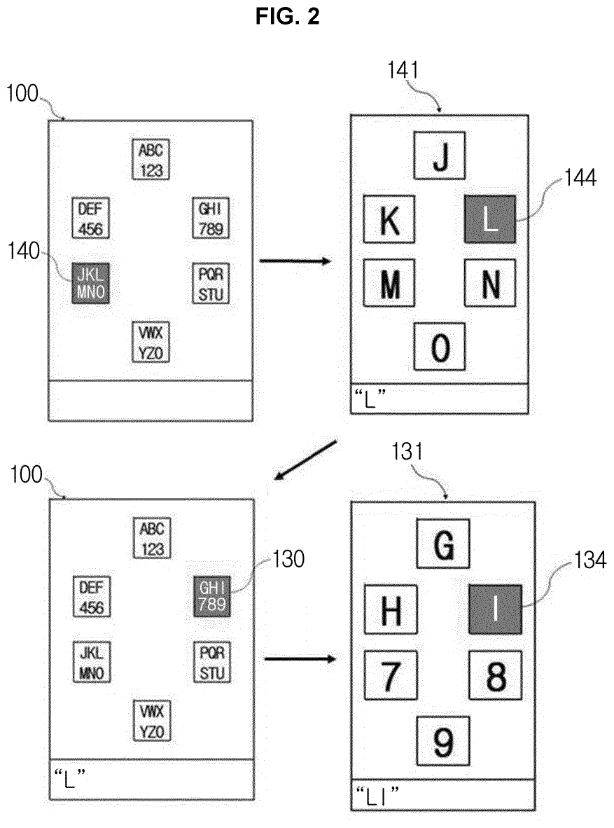

FIG. 2 is a diagram showing a detailed dial in the English input keyboard for a severe patient according to the embodiment of the present disclosure. Referring to FIG. 2, the detailed dial is an English input keyboard which can be selected by the divided display window selected by the patient in FIG. 1.

In FIG. 1, the first divided display 110 includes alphabets and numbers of A, B, C, 1, 2, and 3, and when the first divided display 110 is selected, a first detailed dial 111 including detailed information of the first divided display window 110 may be presented to the patient.

Based on the center of the first detailed dial 111, the first detailed dial 111 may include a first sector 112 positioned at 12 o'clock, a second sector 113 positioned at 10 o'clock, a third sector 114 positioned at 2 o'clock, a fourth sector 115 positioned at 8 o'clock, a fifth sector 116 positioned at 4 o'clock, and a sixth sector 117 positioned at 6 o'clock.

The first detailed dial 111 includes the alphabets and numbers of A, B, C, 1, 2, and 3. Accordingly, the first sector 112 may include information on “A”, the second sector 113 may include information on “B”, the third sector 114 may include information on “C”, the fourth sector 115 may include information on “1”, the fifth sector 116 may include information on “2”, and the sixth sector 117 may include information on “3”.

Similarly, the detailed dial may include a second detailed dial 121 including character information D, E, F, 4, 5, and 6 included in the second divided display window 120, a third detailed dial 131 including character information G, H, I, 7, 8, and 9 included in the third divided display window 130, a fourth detailed dial 141 including character information J, K, L, M, N, and O included in the fourth divided display window 140, a fifth detailed dial 151 including character information P, Q, R, S, T, and U included in the fifth divided display window 150, and a sixth detailed dial 161 including character information V, W, X, Y, Z, and O included in the sixth divided display window 160.

Similarly to the first detailed dial 111, based on the center, the second detailed dial 121 to the sixth detailed dial 161 may include may include first sectors 122, 132, 142, 152, and 162 positioned at 12 o'clock, second sectors 123, 133, 143, 153, and 163 positioned at 10 o'clock, third sectors 124, 134, 144, 154, and 164 positioned at 2 o'clock, fourth sectors 125, 135, 145, 155, and 165 positioned at 8 o'clock, fifth sectors 126, 136, 146, 156, and 166 positioned at 4 o'clock, and sixth sectors 127, 137, 147, 157, and 167 positioned at 6 o'clock.

In the sixth divided window, “0” may be used as a number “0”, and “.” inside “0” may be used a number “0” or a period “.”.

In the embodiment, six divided display windows including alphabets or numbers are disposed. Accordingly, six detailed dials corresponding thereto may also be provided.

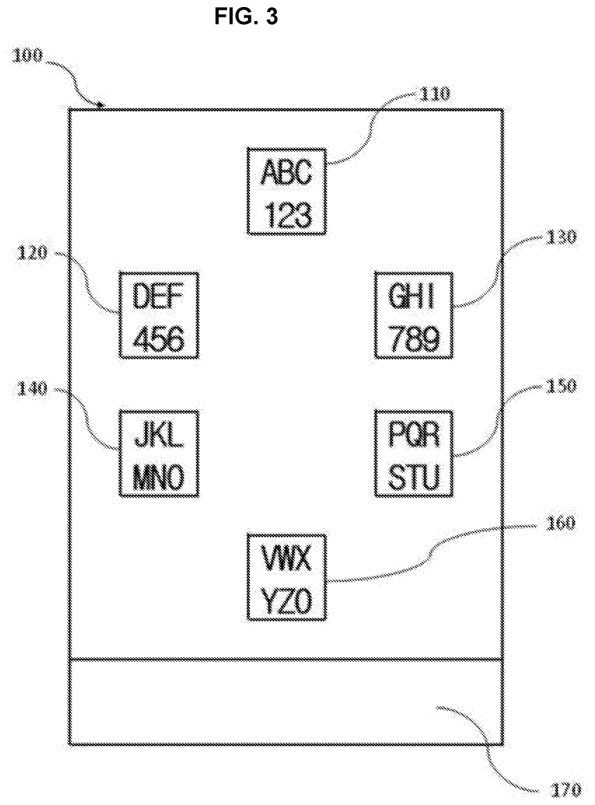

FIG. 3 is a diagram illustrating a process of inputting a character in the English input keyboard for a severe patient according to the embodiment of the present disclosure, and illustrates a process of inputting a single letter using the English input keyboard.

First, the conversation partner (guardian) of the patient presents the integrated dial 100 which is the first English input keyboard, as a location where the patient can see the English input keyboard for communication with the patient. The patient gazes at the divided display window inside the integrated dial 100 including a word or a number corresponding to a beginning of the intended word by moving the pupil to express the intended word. At this time, the guardian observes the movement of the pupil of the patient and recognizes the divided display window determined to be gazing by the patient.

For example, when a letter of “LIKE” is input, the patient will gaze at the fourth divided display window 140 at 8 o'clock including “L”, and the guardian presents the fourth detailed dial 141 including the detailed information of the corresponding fourth divided display window 140 to the patient. The patient will gaze again at the third sector 144 at 2 o'clock where “L” is positioned in the fourth detailed dial 141, and the guardian sees the patient's pupil movement and writes “L” in the character input unit 127. The input process for one alphabet may be performed through the above-described process.

Subsequently, the entire dial 100 may be presented to the patient again. The patient will move the pupil to gaze at the third divided display window 130 at 2 o'clock among the multiple divided display windows disposed on the integrated dial 100. When it is determined that the patient has gazed at the third divided display window 130, the guardian presents the third detailed dial 131 including the character information of the third divided display window 130 to the patient.

The patient will again gaze at the third sector 134 where “I” is located in the third detailed dial 131, and the guardian sees the patient's pupil movement and writes “I” in the character input unit 138. Accordingly, “I” is combined with ‘L’ previously written, and thus, “LI” can be written. By repeating the same process for “K” and “E”, and thus, the word such as “LIKE” is completed.

As described above, in the English input keyboard of the present disclosure, based on the input method of the keyboard, desired characters of the patient are sequentially selected by the guardian according to the intention of the patient, and the selected character is input by the guardian. Accordingly, the word intended by the patient is completed, and thus, quick communication between the patient and the guardian can be achieved.

In the embodiment, considering that the total of the English alphabet and the Arabic numeral is 36, windows which can be selected in six directions based on the center of the English input keyboard are disposed to better recognize the gaze direction of the patient's pupil. That is, since the number of integrated dials and the number of windows disposed inside the detailed dial interlocked therewith are the same as each other, the patient can keep the movement of the pupil the same on the two dials. Accordingly, the guardian is easy to ascertain the position of the pupil of the patient, and the character intended by the patient can be more accurately selected.

In the embodiment, a method is used, in which the guardian presents an English input keyboard to a patient, an alphabet or numeric window selected by the patient is recognized by the guardian, and the corresponding English input keyboard is presented to the patient again. The English input keyboard presented to the patient may be made of paper, or may be made of lightweight plastic for durability.

In addition, the present disclosure may be provided in the form of an application installed on a smart phone or pad. In this case, the integrated screen is presented to the patient in the form of a display, the patient can gaze at a divided display window including the intended alphabet or number on the screen, and the guardian ascertains the position of the pupil of the patient and touches the corresponding divided display window. In the divided display window, the corresponding detailed dial is stored in advance, and thus, the alphabet or number included in the selected detailed dial can be disposed on the screen at a predetermined angle based on the center of the screen. When the patient gazes at the intended alphabet or number through the movement of the pupil, the character appears on a lower end or a specific area of the screen as the conversation partner of the patient touches the corresponding alphabet or number. When a smart phone application is used, the gaze direction of the patient is recognized by a built-in camera, and the character intended by the patient can be displayed through the integrated dial and the detailed dial.

Subsequent processes may be performed in the same manner as described above, a word or a sentence by intended by the patient is expressed by combining the alphabets and numbers selected by the patient, and thus, the guardian may smoothly communicate with the patient.

Hereinbefore, the present disclosure is mainly described with respect to the preferred embodiment, but this is merely an example and does not limit the present disclosure, and a person skilled in the field to which the present disclosure belongs may find that various modifications and applications not illustrated above are possible without departing from the essential characteristics of the present disclosure. For example, each component specifically illustrated in the embodiment of the present disclosure can be implemented by modification. Moreover, differences related to these modifications and applications should be construed as being included in the scope of the present disclosure defined in the appended claims.

The English input keyboard for severe patients according to the embodiment of the present disclosure can be used for a patient who can move his/her eyes of physical movements of the patient, and the patient can quickly and accurately express an intention by staring at the eyes without using an expensive device.

The English input keyboard of the present disclosure can be provided in a state of being displayed on a screen on a portable smartphone or pad, and each screen may be provided with multiple English input keyboards. Accordingly, a method suitable for the patient is selected so that a pseudo expression is possible, and the pseudo expression is possible even when the patient moves.

The English input keyboard of the present disclosure can be used voluntarily or assisted by a conversation partner depending on physical ability of a user, and can be applied to a patient with cognitive abilities corresponding to a normal level and literacy.

What is claimed is:

1. An English input keyboard for a severe patient, comprising:
an integrated dial in which all alphabets and Arabic numbers used in English are disposed to be divided into a predetermined number in multiple divided display windows; and
a detail dial in which an alphabet and number included in the divided display window are disposed in one detail dial,
wherein the divided display window is disposed to have a predetermined angle inside the integrated dial, a plurality of sectors including the alphabet or number included in the divided display window are disposed in the detail dial to have a predetermined angle, and the number and positions of divided display windows inside the integrated dial and the number and positions of sectors inside the detailed dial are the same as each other, and
a conversation partner of the patient presents the integrated dial to the patient, ascertains the divided display window in the integrated dial corresponding to a direction in which a pupil of the patient gazes, presents the detail dial including information in the divided display window to the patient, generates a word or a sentence by repeating a series of processes inputting the alphabet or the number of the sector corresponding to the direction in which the pupil of the patient gazes, and communicates with the patient.

2. The English input keyboard for a severe patient of claim 1, wherein the integrated dial further includes a character input window where the conversation partner of the patient inputs the alphabet or number selected by the patient.

3. The English input keyboard for a severe patient of claim 1, wherein six alphabets, six numbers, or six alphabets and numbers are combined inside the divided display window, and based on a center of the integrated dial, the divided display window includes a first divided display window positioned at 12 o'clock, a second divided display window positioned at 10 o'clock, a third divided display window 130 positioned at 2 o'clock, a fourth divided display window positioned at 8 o'clock, ae fifth divided display window 150 positioned at 4 o'clock, and the sixth divided display window 160 positioned at 6 o'clock.

4. The English input keyboard for a severe patient of claim 3, further comprising:
a first detailed dial in which character information included in the first divided display window is disposed one by one in six directions;
a second detailed dial in which character information included in the second divided display window is disposed one by one in six directions;
a third detailed dial in which character information included in the third divided display window is disposed one by one in six directions;
a fourth detailed dial in which character information included in the fourth divided display window is disposed one by one in six directions;
a fifth detailed dial in which character information included in the fifth divided display window is disposed one by one in six directions; and
a sixth detailed dial in which character information included in the sixth divided display window is disposed one by one in six directions.

5. The English input keyboard for a severe patient of claim 4, wherein the first detail dial to the sixth detail dial include a first sector which is positioned at 12 o'clock based on a center and includes one character, a second sector which is positioned at 10 o'clock based on the center and includes one character, a third sector which is positioned at 2 o'clock based on the center and includes one character, a fourth sector which is positioned at 8 o'clock based on the center and includes one character, a fifth sector which is positioned at 4 o'clock based on the center and includes one character, and a sixth sector which is positioned at 6 o'clock based on the center and includes one character.
